(19) United States
(12) Patent Application Publication
(10) Pub. No.: US 2026/0191608 A1
(43) Pub. Date: Jul. 9, 2026

(54) Systems, Methods and Devices for Remote and Autonomous Control of a Flexible Endoscope

(71) Applicants: Andrew Gumbs, Paris (FR); Luca Milone, New York, NY (US)

(72) Inventors: Andrew Gumbs, Paris (FR); Luca Milone, New York, NY (US)

(21) Appl. No.: 19/129,297

(22) PCT Filed: Nov. 13, 2023
(86) PCT No.: PCT/IB2023/000725

§ 371 Date: May 12, 2025

Related U.S. Application Data
(60) Provisional application No. 63/424,763, filed on Nov. 11, 2022.

(51) Int. Cl. A61B 34/32; A61B 1/00; A61B 1/005; A61B 1/018; A61B 34/20; A61B 34/30

(52) U.S. Cl. CPC A61B 34/32; A61B 1/00006; A61B 1/000094; A61B 1/00087; A61B 1/00096; A61B 1/00097; A61B 1/00149; A61B 1/0016; A61B 1/0051; A61B 1/009; A61B 1/018; A61B 34/20; A61B 2034/2063; A61B 2034/302

(57) ABSTRACT

Systems, methods and devices are provided for robotic endoscopy using a robotically-controlled dual-arm flexible endoscope, with a first robotic arm holding and controlling a robotically-controlled endoscope handle manipulator, and a second robotic arm holding and controlling an endoscope actuator for advancement and retraction of a distal tip of the endoscope. Each robotic arm is capable of a multitude of movements that essentially mimic the function of human arms and hands, allowing the flexible endoscope to be repositioned, twisted and torqued as a human operator would in order to change the direction of the flexible endoscope as it moves through non-linear organic structures within a body cavity of an animal or human. The robotic arms may be mounted on a single base and controlled via a single controller mechanism or on separate bases with separate controller mechanisms to potentially allow for increased angles of movement and positioning for each robotic arm.

### BACKGROUND

### Field of the Invention

Systems, methods and devices provided herein relate to a remote-controlled endoscope, and more specifically to a dual-arm robotic endoscope and embedded sensors for performing endoscopic procedures both automatically and autonomously.

### Related Art

Endoscopic surgery is now a leading option for performing minimally-invasive surgery, as it allows for the use of a small incision to insert a scope and a flexible tube with multiple tools that allow a surgeon to perform a plurality of procedures. The tools typically include a camera and light to allow the surgeon to visualize an area inside a body without making large incisions. The functionality of each endoscope is often so complex that it requires multiple operators or specialized controls such as foot pedals to allow a surgeon to carefully control the movement and operation of the tools all at the same time.

The initial field of endoscopy, which traditionally only involved inserting the scope and tube into an existing orifice, has now expanded to include multiple disciplines, including surgery and interventional radiology, and the types of procedures which can be completed via endoscopy are rapidly expanding as well. As the types of procedures performed become more complex, interventional doctors must now learn multiple disciplines in order to choose the best method to solve a problem.

Recent improvements have attempted to automate some of the control and movement of the flexible endoscopes to reduce the complexity of the procedure for a surgeon or allow remote-controlled operation. This includes the use of actuators and manipulators to allow remote controlled operation of an endoscope to control all of the features of a flexible endoscope, from the depth within the body to the direction of movement, operation of tools, camera, lighting, etc. Several of these improvements are described in U.S. Pat. Nos. 8,409,080 and 9,706,907, incorporated herein by reference.

However, improvements to the ability to move and control the flexible endoscope are still needed, particularly as the types of procedures become more complex and are more advantageously performed via remote control. As options for the remote control improve, additional options for autonomous control of the endoscopes may be considered.

### SUMMARY

Embodiments described herein provide for systems, methods and devices for robotic endoscopy using a robotically-controlled dual-arm flexible endoscope, with a first robotic arm holding and controlling a robotically-controlled endoscope handle manipulator, and a second robotic arm holding and controlling an endoscope actuator for advancement and retraction of a distal tip of the endoscope. Each robotic arm is capable of a multitude of movements that essentially mimic the function of human arms and hands, allowing the flexible endoscope to be repositioned, twisted and torqued as a human operator would in order to change the direction of the flexible endoscope as it moves through non-linear organic structures within a body cavity of an animal or human. The robotic arms may be mounted on a single base and controlled via a single controller mechanism or mounted on separate bases with separate controller mechanisms to potentially allow for increased angles of movement and positioning for each robotic arm.

Manual control of knobs of the endoscope handle may be accomplished via a foot pedal, and the use of robotically-controlled guide wires may also benefit from a seat to allow each foot to control separate functions via separate foot pedals. Alternatively, a console may be constructed to provide for better control options.

The controller mechanisms are also capable of being operated remotely for tele-manipulation of complex endoscopic procedures, as well as being capable of performing automatic actions by using the combination of sensors and tools on the flexible endoscope to automatically execute specified functions, via computer-assisted surgery and intervention or image-guided surgery and intervention. Additionally, artificial intelligence surgery is also possible with this device and system, where the controller or controllers allow for autonomous navigation and actions via reinforcement learning algorithms and machine learning.

The robotically-controlled flexible endoscope may also be adaptable for use with disposable endoscopes and grips and non-traditional endoscopes that have wider channels to allow for thicker guide wires and more complex functionalities at the endoscope tip. These non-traditional endoscopes may be enhanced to improve the autonomous functionality, such as including a shape-sensing fiber to measure the shape and length of the endoscope.

In one embodiment, a robotically-controlled dual-arm flexible endoscope comprises: a flexible endoscope with a shaft, a proximal handle and a distal tip; a first robotic arm secured with the proximal handle and configured to move the proximal handle of the flexible endoscope; an endoscope handle manipulator connected with the first robotic arm and proximal handle, and configured to control the flexible endoscope; a second robotic arm with an endoscope actuator configured to secure the distal tip and advance or withdrawal the flexible endoscope into or from a body cavity, respectively; and a controller in communication with the first robotic arm and second robotic arm to control the movement and position of each robotic arm, the handle manipulator and the endoscope actuator.

In another embodiment, a method of performing an endoscopic procedure with a robotically-controlled flexible endoscope comprises the steps of: controlling, via a controller, the movement and function of an endoscope handle manipulator connected with a proximal handle of a flexible endoscope via a first robotic arm; controlling, via a controller, the movement and function of an endoscope actuator secured with a distal tip of the flexible endoscope via a second robotic arm; and performing the endoscopic procedure by via movement and control of the flexible endoscope by the controller.

In a further embodiment, a method of screening for a disease using an autonomous flexible endoscope comprises: inserting a flexible endoscope into a body cavity using a first robotic arm and second robotic arm controlled by a controller to adjust a movement and function of the flexible endoscope; utilizing a plurality of sensors disposed along the endoscope and at a distal end to identify pathologies which indicate a presence of disease; conducting at least one diagnostic test using the plurality of sensors and at least one tool disposed at the distal end of the endoscope to determine the presence of the disease; and performing at least one endoscopic surgical procedure to treat the identified disease.

Other features and advantages of the present invention will become more readily apparent to those of ordinary skill in the art after reviewing the following detailed description and accompanying drawings.

### BRIEF DESCRIPTION OF THE DRAWINGS

The structure and operation of the present invention will be understood from a review of the following detailed description and the accompanying drawings in which like reference numerals refer to like parts and in which:

FIG. 1 is an illustration of a robotically-controlled dual-arm flexible endoscope, according to an embodiment of the invention;

FIG. 2 is an illustration of the robotically-controlled dual-arm flexible endoscope with separate bases for each arm, according to one embodiment of the invention;

FIG. 3 is an illustration of an endoscope handle manipulator connected with a first robotic arm, according to an embodiment of the invention;

FIG. 4 is an illustration an endoscope actuator connected with a second robotic arm, according to one embodiment of the invention;

FIG. 5 is an illustration of a sheath or condom with a plurality of location-sensing devices configured thereon, according to one embodiment of the invention;

FIG. 6A is a side-view illustration of an endoscope with a monorail formed along an inner circumference of the scope shaft for deploying a tool, according to one embodiment of the invention;

FIG. 6B is a top-view illustration of an endoscope with a monorail formed along an outer circumference of the scope shaft for deploying the tool, according to one embodiment of the invention;

FIG. 6C is a cross-sectional view of the scope shaft illustrating the monorail disposed along an inner circumference of the shaft, according to one embodiment of the invention;

FIG. 6D is a magnified view illustration of a distal portion of the scope shaft with the monorail showing the tool in a deployed position, according to one embodiment of the invention;

FIG. 7A is an illustration of a scope shaft with light sources and sensors located inside of an intestinal cavity, according to one embodiment of the invention;

FIG. 7B is a magnified view illustration of the light sources and sensors positioned on the scope shaft to detect reflected light from a tissue wall, according to one embodiment of the invention;

FIG. 7C is a cross-sectional views illustration of a plurality of light sensors disposed around an outer circumference of the scope shaft, according to one embodiment of the invention;

FIG. 7D is a cross-sectional views illustration of a plurality of light sensors disposed around an outer circumference of the scope shaft, according to one embodiment of the invention;

FIG. 8 is an illustration of a system for performing an endoscopic procedure remotely or autonomously using the robotically-controlled dual-arm flexible endoscope, according to one embodiment of the invention;

FIG. 9 is an illustration of a method for performing an endoscopic procedure using the robotically-controlled dual-arm flexible endoscope, according to one embodiment of the invention; and

FIG. 10 is a block diagram illustrating an example wired or wireless processor enabled device that may be used in connection with various embodiments described herein.

### DETAILED DESCRIPTION

Certain embodiments disclosed herein provide for systems, methods and devices for robotic endoscopy using a robotically-controlled dual-arm flexible endoscope, with a first robotic arm holding and controlling a robotically-controlled endoscope handle manipulator, and a second robotic arm holding and controlling an endoscope actuator for advancement and retraction of a distal tip of the endoscope. Each robotic arm is capable of a multitude of movements that essentially mimic the function of human arms and hands, allowing the flexible endoscope to be repositioned, twisted and torqued as a human operator would in order to change the direction of the flexible endoscope as it moves through non-linear organic structures within a body cavity of an animal or human. The robotic arms may be mounted on a single base and controlled via a single controller mechanism or mounted on separate bases with separate controller mechanisms to potentially allow for increased angles of movement and positioning for each robotic arm.

Manual control of knobs of the endoscope handle may be accomplished via a foot pedal, and the use of robotically-controlled guide wires may also benefit from a seat to allow each foot to control separate functions via separate foot pedals. Alternatively, a console may be constructed to provide for better control options.

The controller mechanisms are also capable of being operated remotely for tele-manipulation of complex endoscopic procedures, as well as being capable of performing automatic actions by using the combination of sensors and tools on the flexible endoscope to automatically execute specified functions, via computer-assisted surgery and intervention or image-guided surgery and intervention. Additionally, artificial intelligence surgery is also possible with this device and system, where the controller or controllers allow for autonomous navigation and actions via reinforcement learning algorithms and machine learning.

The ability to perform flexible endoscopy via robotic control and more specifically automatic and autonomous control will provide marked increases in safety and accuracy for both intraluminal and transluminal therapeutic gestures. To robotize a traditional flexible endoscope means the elimination of the shortcomings given by the cumbersome paths of reaching a certain point in a digestive tube or other body cavity with complex anatomy.

After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention as set forth in the appended claims.

### Robotically-Controlled Dual-Arm Endoscope

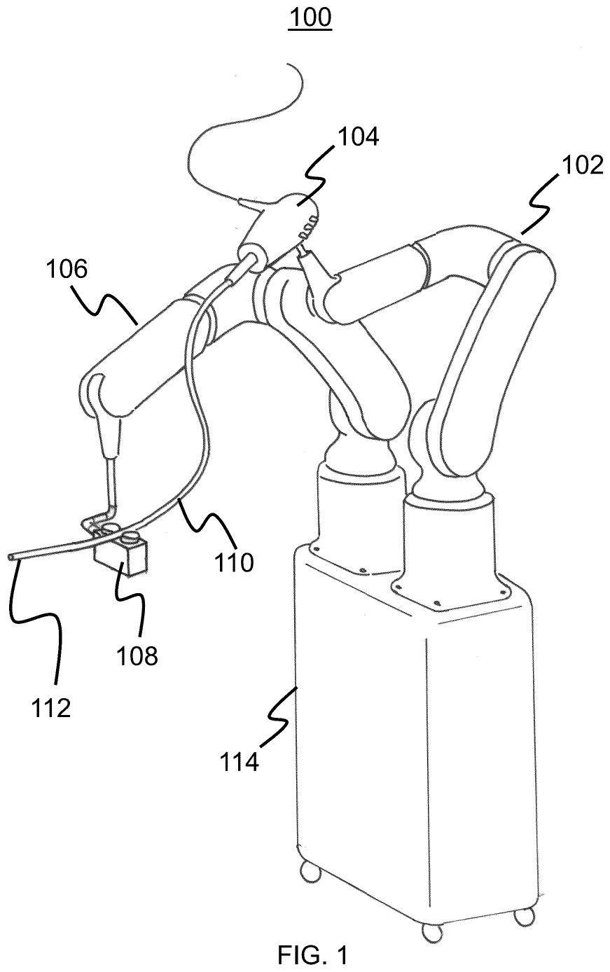

One embodiment of the robotically-controlled dual-arm flexible endoscope is illustrated in FIG. 1, which shows the overall endoscope 100, along with a first robotic arm 102 attached with an endoscope handle manipulator 104 and a second robotic arm 106 attached with an endoscope actuator 108. The flexible endoscope has a shaft 110 extending from the handle manipulator to the distal tip 112 which includes the tools, camera, lighting, etc. used to complete the endoscopic procedure. In this embodiment, a singular base 114 is used for both arms, which may also include the controller and other computer-related equipment to execute the specific functions of the desired procedures.

The first robotic arm 102 controls the location, movement and torque of the endoscope handle manipulator 104 at the proximal end of the flexible endoscope, while the second robotic arm 106 controls the location, movement and torque of the endoscope actuator 108 which is used to translate the scope shaft 110 by advancing or withdrawing the distal tip 112 into a body cavity. Together, the first robotic arm 102 and second robotic arm 106 allow the flexible endoscope to be moved with six degrees of freedom, from rotational movement along an x, y and z axis, as well as translational movement along those three axes. In one embodiment, the actuation of the endoscope handle manipulator 104 may be controlled via a foot pedal, but if guide wires within the scope shaft 110 are robotically controlled, a seat may be incorporated for the operator to operate the manipulator 104 with a right foot pedal and the endoscope actuator 108 with a left foot pedal. Alternatively, a console may be provided to allow for a centralized control module.

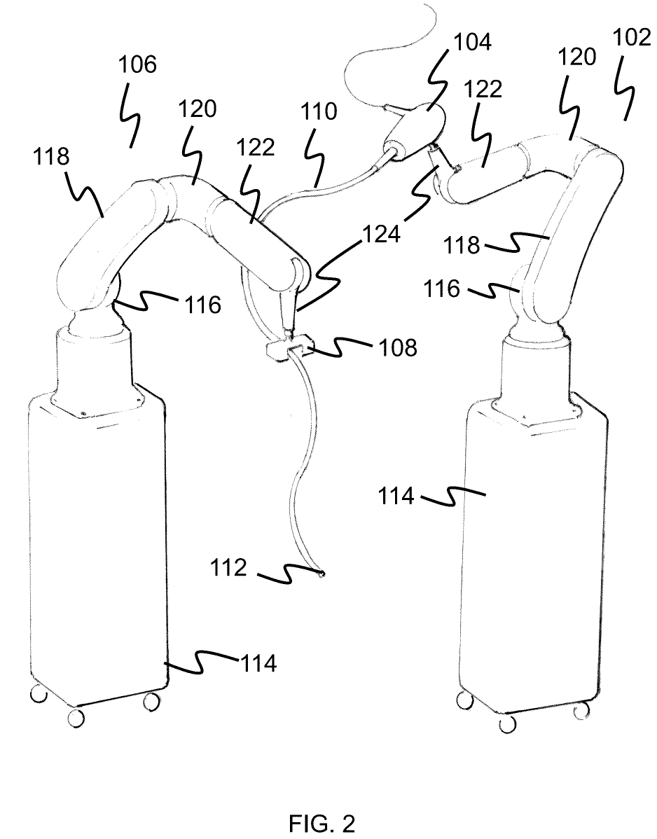

In one embodiment illustrated in FIG. 2, each robotic arm has its own base 114, potentially allowing a greater deal of movement and positioning of the flexible endoscope and offering alternatives for setting up a procedural room. FIG. 2 also provides a more detailed view of the individual components of each robotic arm (102, 106) which provides for movement in essentially any direction in order to mimic the movement of human arms that would otherwise be performing the procedure. The first robotic arm 102 and second robotic arm 106 may be essentially identical in terms of their design and overall movement, and only differ with regard to the attachments at their distal ends where the endoscope handle manipulator 104 is attached with the first robotic arm 102 and the endoscope actuator 108 is attached with the second robotic arm 106. The robotic arms (102, 106) may include a rotating base portion 116, an upper arm extension 118, middle arm extension 120, lower arm extension 122 and attachment extension 124. Each extension portion is rotatably attached with the next to mimic the movement of a human arm with options for torque and twisting movements, from the rotator cuff shoulder joint of the rotating base portion 116 and upper arm extension 118 to the wrist joint represented by the lower arm extension 122 and attachment extension 124.

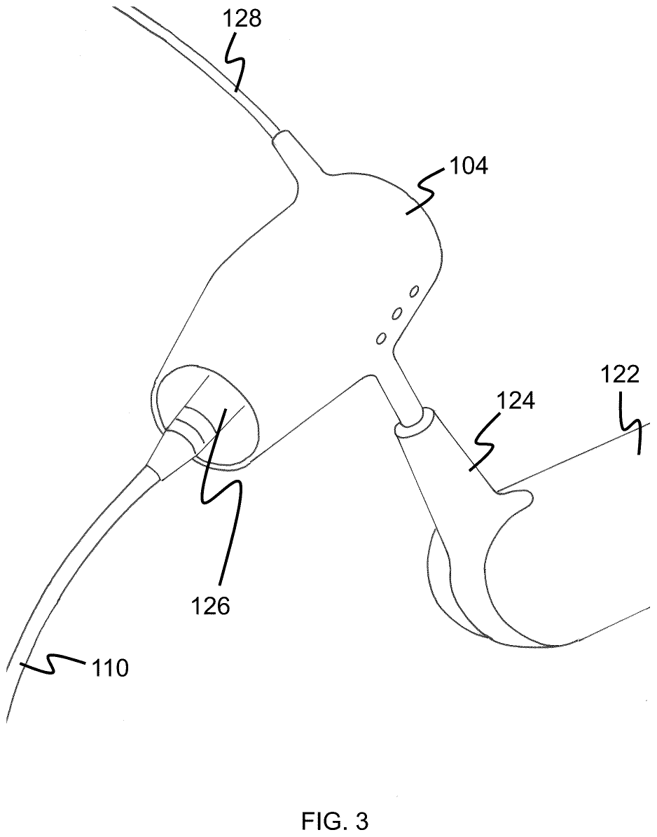

FIG. 3 is a detailed view illustration of the endoscope handle manipulator 104 attached with the first robotic arm 102, according to one embodiment of the invention. The endoscope handle manipulator 104 is attached with the attachment extension 124 that rotates with respect to the adjacent lower arm extension 122, but which may also rotate axially around the length of the attachment extension 124 as well. The manipulator 104 is configured to control the primary operations of the flexible endoscope in terms of the cameras, lighting, tools and any other features located on the distal tip 112. These elements are connected via the scope 126 which extends from the manipulator and becomes the scope shaft 110 that terminates with the distal tip 112 (not shown) where the tools and other elements of the endoscope are located. In one embodiment, a separate connector 128 may be utilized to provide power and output the images and other data from the endoscope to a terminal, monitor or other console used by the operator to perform the endoscopic procedure. However, this may also be communicated through the robotic arm 102 to a controller located in the base 114.

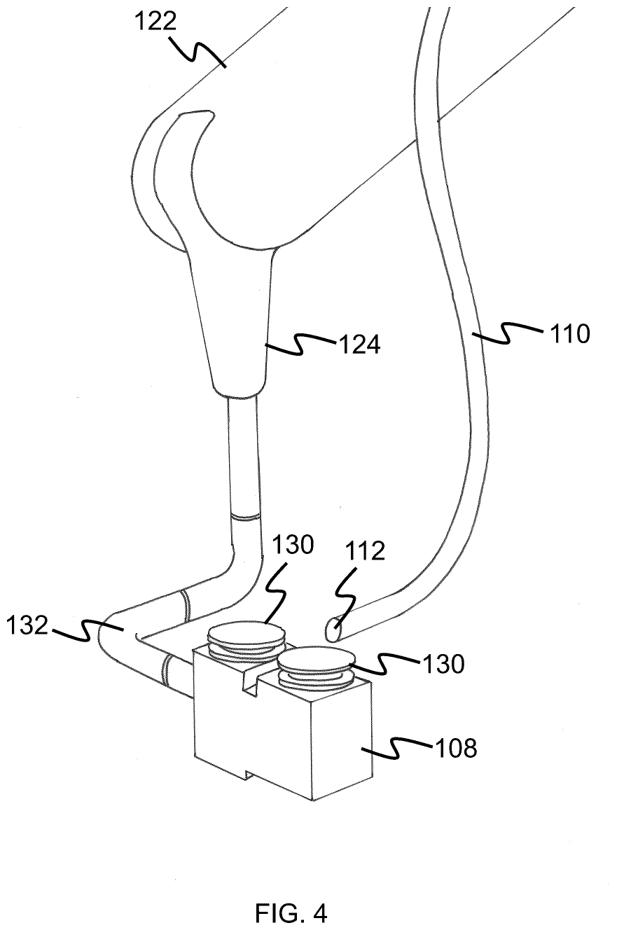

FIG. 4 is a detailed view illustration of an endoscope actuator 108 showing the a pair of axial rollers 130 which are used to translate movement of the shaft 110 to advance or withdrawal the distal tip of the endoscope 112 into, out of or around the body cavity. The attachment extension 124 may be connected with a support arm 132 that maintains the endoscope actuator at a fixed angle relative to the second robotic arm 106. The support arm 132 may also include any needed power and data cables to provide power, control and feedback of the endoscope actuator to and from the operator.

### Remote and Autonomous Control

In one embodiment, the use of one or more controllers and two highly flexible robotic arms provides additional options for improved remote control of an endoscopic procedure including the ability to perform automatic or autonomous functions. Controllers operating each robotic arm can provide detailed feedback and complex data on the movement and control of the flexible endoscope used in conjunction with images and other sensory data obtained from the endoscope tip, which can be processed into recommendations and actions for the movement and operation of the endoscope, either by the operator or automatically by a computing device which is managing both the robotic arms and the flexible endoscope. The operator may be located remotely from the device, such that the endoscope and robotic arms can be tele-manipulated.

Additionally, the robotically-controlled dual-arm flexible endoscope may incorporate artificial intelligence (AI) machine learning algorithms within the controllers and computing devices operating the flexible endoscope and robotic arms in order to develop options for autonomous endoscopic procedures such as colonoscopies, upper endoscopies, bronchoscopes, thorascopies, ctyoscopies, etc. Unlike traditional AI which is used primarily to analyze complex data sets, these algorithms will be used to translate computer vision data, robotic arm controller data and other sensing tools into physical action and movement of the endoscope and robotic arms, even to the point of performing interventional gestures and actions. The learning algorithms will aid in both navigation and execution of various procedures through the aid of the surgeon or operator or completely autonomously without any human intervention.

### Expanded Functionality

The design of the improved robotically-controlled dual-arm flexible endoscope also allows for the incorporation of several additional improvements to the equipment and function of the flexible endoscope. For example, the robotically-controlled dual-arm flexible endoscope allows for the use of inexpensive, disposable endoscopes, which can be made with wider channels to allow for thicker guide wires that provide more complex functionalities at the distal tip 112.

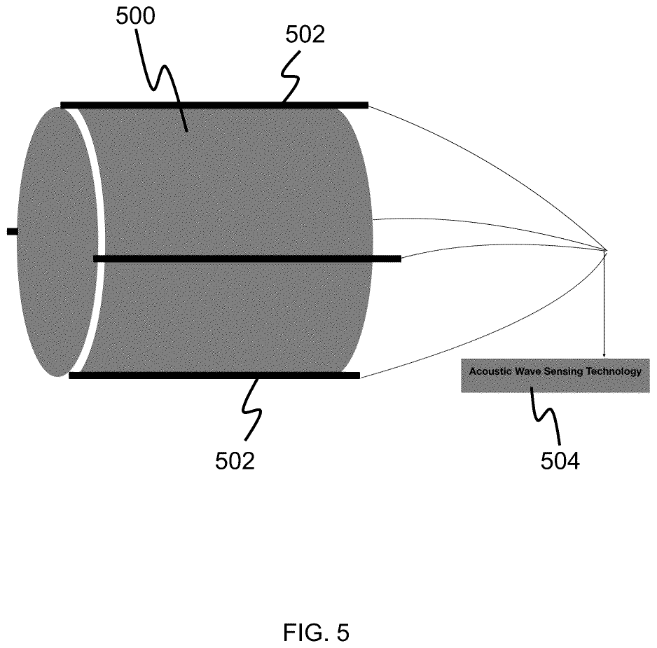

Additionally, advanced technology such as fibers with shape-sensing technology to accurately identify the location and shape of the endoscope within the body cavity can be incorporated into the scope, although additional disposable elements such as a sheath or condom to fit over the endoscope shaft to retain the fiber may be needed until the shape sensing fibers can be incorporated into the scope shaft 110 and the fiber optic ends at the distal tip 112. FIG. 5 is an illustration of one embodiment of a sheath (condom) 500 with wires 502 in four quadrants configured to be placed over an existing endoscope tip and attached to a system 504 to interpret acoustic signals. Reinforcement learning algorithms will be used to permit autonomous and safe endoscopies by triangulating signals from the four quadrants of the endoscope tip. Unlike traditional methods of guidance using image interpretation and computer vision, algorithms analyzing acoustic signals have the advantage of utilizing less data, which should speed up safe autonomous actions to provide for real life applicability. For reference, see Schaufler, Anna, Illanes, Alfredo, Maldonado, Ivan, Boese, Axel, Croner, Roland and Friebe, Michael. “Surgical Audio Guidance: Feasibility Check for Robotic Surgery Procedures” Current Directions in Biomedical Engineering, vol. 6, no. 3, 2020, pp. 571-574, https://doi. org/10.1515/cdbme-2020-3146 and Mahmoodian, Naghmeh; Schaufler, Anna; Pashazadeh, Ali; Boese, Axel; Friebe, Michael; et al. Proximal detection of guide wire perforation using feature extraction from bispectral audio signal analysis combined with machine learning. Computers in Biology and Medicine; Oxford Vol. 107, (Apr 2019): 10-17. DOI:10.1016/j.compbiomed.2019.02.001.

Additional portions of the device may also be disposable to avoid wear and tear and any potential contamination from reusable components which contact portions of the endoscope that enter the body cavity, such as grips on the axial rollers which are located on the endoscope actuator and translate the shaft into or out of the body cavity.

### Rail Delivery System

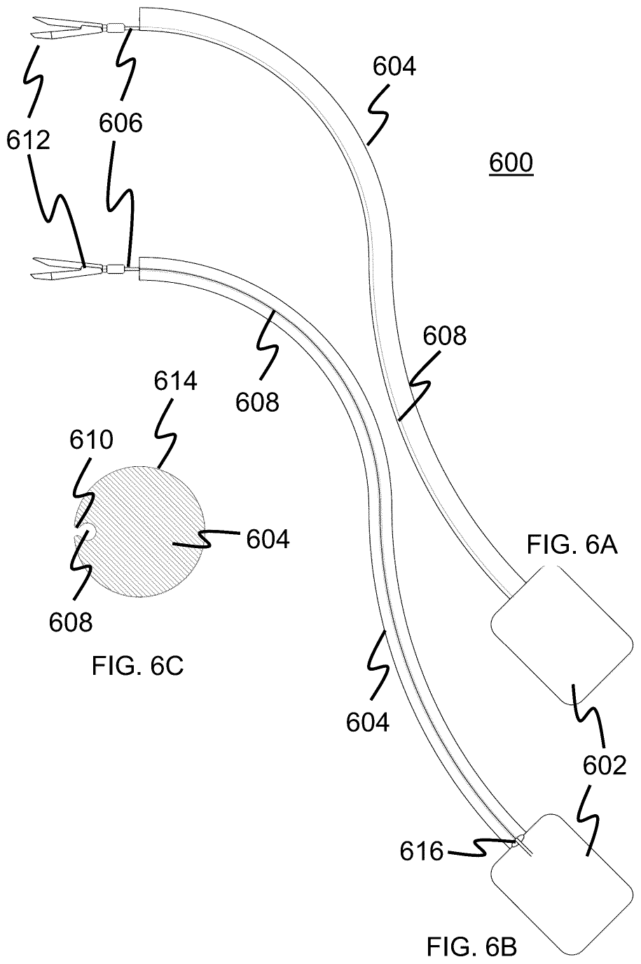

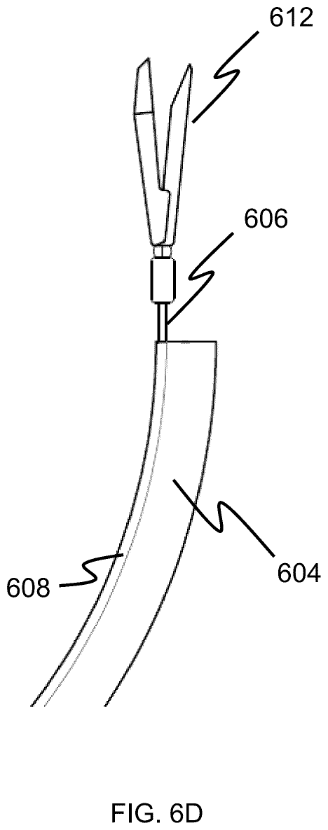

FIGS. 6A-6D illustrate a rail delivery system where a monorail is disposed within the endoscopic shaft in order to more easily deliver one or more tools or instruments for use in an endoscopic procedure. FIG. 6A is a side-view illustration of an endoscope 600 with a manipulator 602 and a scope shaft 604 shown in a transparent view to illustrate a monorail 608 positioned along the length of the scope shaft 604 along an inner circumferential wall 610 of the scope shaft 604, as shown more clearly in FIG. 6C. In this embodiment, the monorail 608 is used to deliver a lateral stapler 612, which is shown in a deployed configuration which is still attached to the monorail 608 via a connector 606 which allows the stapler 612 to rotate into position for use but also be withdrawn along the monorail 608 if needed. The manipulator 602 can either operate and control the tool 612 in its entirety or provide a separate controller or handle for partial or fully manual operation.

In another embodiment illustrated in FIG. 6B, the monorail 608 could be used to deliver a tool along an outer circumference 614 of the shaft 604 using a separate insertion point 616 to attach the tool 612 via the connector 606. The monorail 608 could be similarly shaped as that shown in FIG. 6C to protrude into the circumference of the shaft and allow the tool to slide along the hollowed-out groove created by the monorail 608.

FIG. 6D is a magnified view illustration of a distal portion of the scope shaft 604 with the monorail 608 showing the tool 612 in a deployed position connected with the monorail 608 via the connector 606. The monorail 608 allows the tool 612 to be more easily deployed through a flexible endoscope such as that illustrated herein, and preserves space within the shaft 604 for other tools and instruments. It may also allow different types of tools to be utilized which might otherwise be difficult to insert traditionally, such as a circular stapler or a vessel sealing device used to seal gastrointestinal walls or remove polyps and other tumors.

### Sensor-Embedded Sheath

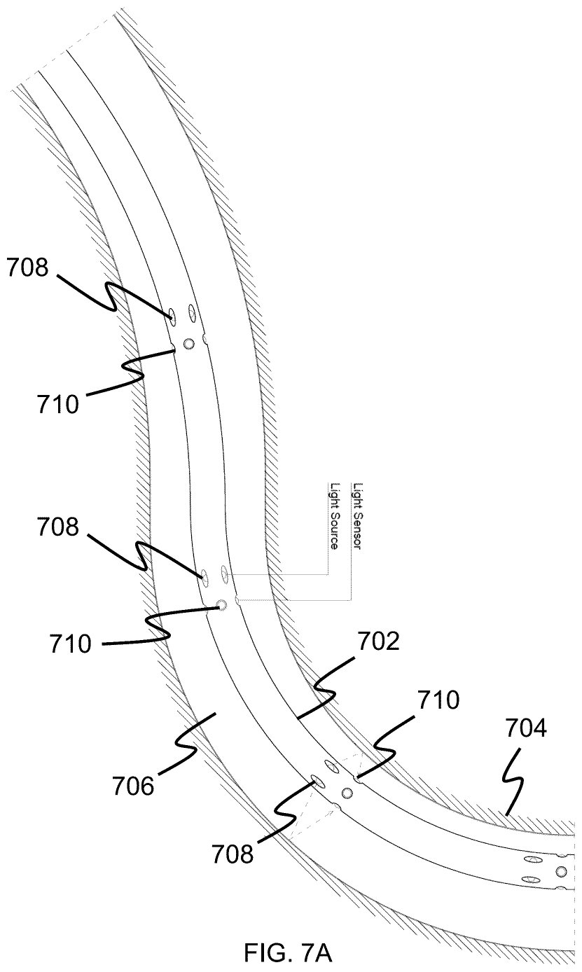

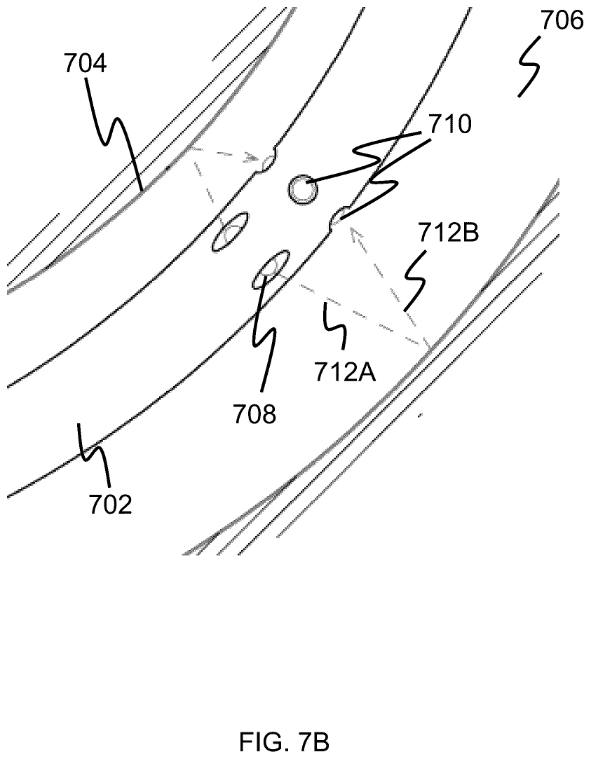

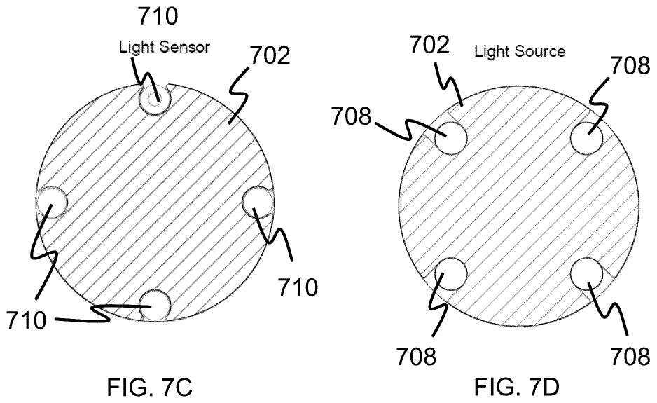

In one embodiment, the endoscope shaft may be embedded with various types of sensors used to detect the location and other navigational data which can be used to position and move the scope and tool to particular positions or to avoid problematic areas. FIG. 7A illustrates one embodiment of a scope shaft 702 positioned between an intestinal wall 704 of an intestinal cavity 706, such as during an anoscopy. Although a variety of sensors could be utilized, one embodiment illustrated in FIG. 7B is the use of a combination of light sources 708 and optical sensors 710 disposed along the length of the shaft 702 which work in combination to transmit light 712B outward from the light sources 708 along the length of the shaft, which then reflects light 712B off of the surrounding tissue walls 704 that is then detected by the adjacent sensors 710.

The sensors 710 can then use the reflected light 712B to detect not only the distance, location and position of the shaft relative to the tissue walls, but also use multispectral imaging, narrow band imaging, hyperspectral imaging and multispectral imaging to enhance navigation or detect different wavelengths being reflected from adjacent tissue to determine if there are areas of the tissue that may need further investigation or treatment as part of an autonomous diagnosis of pathologies during the endoscopic procedure. In one embodiment, the robotically-controlled dual-arm flexible endoscope can be attached with an endoscopic ultrasound to enable autonomous endoscopic echography. During upper endoscopy, for example, the endoscope can perform autonomous surveillance of organs such as the pancreas and be further configured to perform autonomous fine needle aspiration of suspicious lesions.

In one embodiment, the light sources are light-emitting diodes (LEDs) and the optical sensors 710 are cameras which may be used as part of an autonomous echo endoscopy. Light can be emitted circumferentially at locations which substantially cover the outer circumference of the shaft at a particular point, and paired sensors will detect the light reflected off of nearby tissue to enable a calculation of a distance of the shaft from surrounding tissue. In one embodiment, the light sources and optical sensors are placed every few centimeters and pulsed at different times so that the sensors and connected computing devices can calculate which portion of the shaft is at which portion of a body cavity, as well as whether the shaft may be touching a body cavity wall.

As noted above, the use of the sensors will allow for autonomous echo endoscopy by allowing the scope and connected tools to autonomously navigate via and screen for pathologies such as pancreatic cancer. Various hardware and software products are capable of analyzing video data, like video surgomics, which is a field which uses computer vision, machine learning and multimodal data during endoscopic interventional procedures to provide predictions based on data collected therein.

FIG. 7C illustrates a cross-section of the scope shaft 702 taken at the position of a bank of light sensors 710 disposed at different locations around the circumference of the shaft 702. Similarly, FIG. 7D illustrates a cross-section of the scope shaft 702 taken at the position of a bank of light sources 708 disposed at different locations around the circumference of the shaft 702. As shown previously in FIGS. 7A and 7B, the light sources 708 and light sensors 710 may be placed adjacent to one another so that the light sources transmit light in the vicinity of the light sensors which can then be reflected to the light sensors for processing of the relevant information, such as distance from tissue, type of tissue, abnormal colors, textures, depths or patterns, etc. which may be evident of a pathology.

In another embodiment, different sensors will be placed at different section of the shaft depending upon the data which needs to be collected from each location along the shaft. For example, the light sources 708 and optical sensors 710 or other location-type sensors may be positioned along the length of the shaft 702 to determine the position and location of the shaft, while other types of sensors may be placed toward the distal end of the shaft 702 to more accurately identify the type of tissue, organ or abnormalities therein to aid in the performance of any type of relevant endoscopic procedure.

In one embodiment, the sensor may be a vibroacoustic sensor which is disposed within the wall of the scope shaft to use as part of an audio-based guidance system known as Surgical Audio Guidance (SURAG). The vibroacoustic sensor is a passive, plug-and-play sensing solution that may be attached to a proximal end of the vibroacoustic sensor outside of a patient's body, where it will be integrated into an endoscope handle. The vibroacoustic sensor may be a string surrounded by fluid in a thin flexible tube, where the fluid (such as oil or water) is used to transmit the vibration detected by the string). The vibroacoustic sensor may therefore be covered and embedded within the shaft to provide insulation to dampen false vibrations.

### Anoscopy

The robotically-controlled dual-arm flexible endoscope may be integrated with various types of scopes for performing a variety of different procedures. In one embodiment, the robotically-controlled dual-arm flexible endoscope may be integrated with an anoscope to perform anoscopy and provide treatment in anal canal or rectum. In one example, a trident anoscope device, such as that described in U.S. Pat. No. 10,729,318, may be incorporated with the robotically-controlled dual-arm flexible endoscope, including the use of flexible ablation wires to provide intraluminal treatments inside the gastrointestinal tract or the bronchial tree. Ablation wires with integrated temperature control will allow for safe ablation of hollow-viscus structures without causing perforation, and temperature control would also enable a dramatic reduction in post-ablation hemorrhage after intra-bronchial ablations because specifically-selected lower temperatures could be utilized which are sufficient to perform a desired treatment but low enough to prevent perforation and/or post-operative hemorrhage.

In another embodiment, a hollow ablation needle could be used for injection of immunotherapy into a post-ablation region. In some instances, post-ablation immunologic factors released after an ablation have been shown to harbor the potential to create tumor vaccines. By injecting immunotherapy directly into the tumor that has just been ablated, a vaccine against tumors could be developed. This has the potential to reduce local recurrence as well as distant recurrence of these pathologies.

### Computer Vision

To provide for fully-autonomous endoscopic procedures, the use of automated devices like the robotically-controlled dual-arm flexible endoscope will need to be combined with computer vision and other machine learning tools to aid in navigation, diagnostics and treatments during any type of endoscopic procedure. As noted above, the use of sensors along the scope shaft and at the distal end of the scope shaft will provide additional imaging data (including narrow band imaging, hyperspectral imaging and multispectral imaging) that can be used for navigation, diagnostics and treatment. The incorporation of biophotonics with computer vision could further enhance the algorithm's ability to diagnose pathologies.

In one embodiment, computer vision and diagnostic enhancements may be combined with the robotically-controlled dual-arm flexible endoscope and the aforementioned anoscope to develop a guaiac-based fecal occult blood test. In this embodiment, a clear plastic anoscope may be utilized to allow computer vision to determine if a patient is guaiac positive by analyzing whether a stool sample changes color, indicating the presence of iron and therefore blood in the stool. A positive test result during anoscopy would then trigger an automatic colonoscopy which could be performed by the robotically-controlled dual-arm flexible endoscope already in place for the anoscopy. An ablation or other trident-like treatment device can then be introduced into the anoscope to perform the colonoscopy and automatically perform any needed treatment.

### System Overview

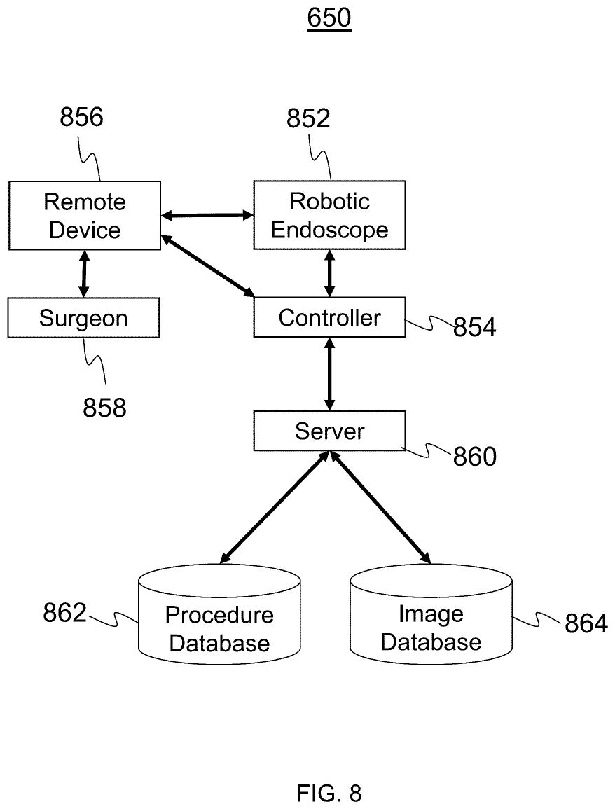

FIG. 8 is an illustration of a system 850 for performing an endoscopic procedure using the robotically-controlled dual-arm flexible endoscope and remotely-located devices from either a remote user or an autonomous system. In this embodiment, the robotic endoscope 852 may be operated by a controller 854 which can be controlled by either a surgeon 858 operating a remote device 856, or autonomously via a computing device like a server 860 running software and performing endoscopic operations via data obtained from a procedures database 862 and image database 864. For example, the flexible endoscope may be sending images from the camera on the distal tip 112 to the server 860, which then analyzes the images in comparison with images in the image database 864 to determine potential movement and operation of the endoscope and related tools.

### Method of Performing an Endoscopic Procedure

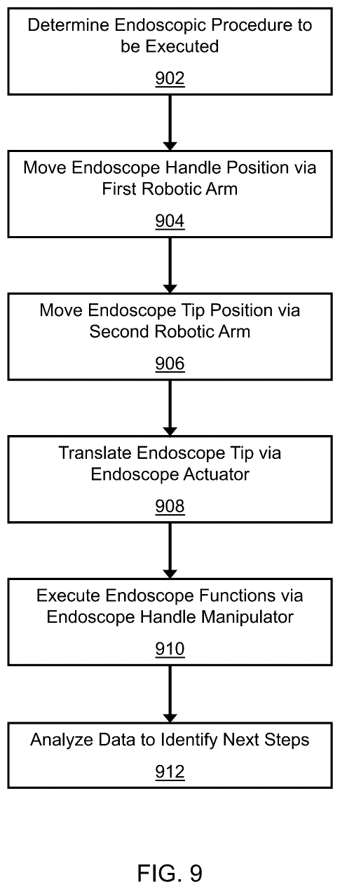

FIG. 9 illustrates an exemplary method for performing an endoscopic procedure using the robotically-controlled dual-arm flexible endoscope. In step 902, a particular type of endoscopic procedure is selected, and in step 904, the first robotic arm moves the endoscope handle manipulator into a location, position and angle needed for optimal performance and function of the desired movement and operation. In step 906, the second robotic arm moves the endoscope actuator into a location, position and angle needed for optimal performance of the desired movement and operation. With both arms in place, in step 908, the distal tip of the endoscope be translated into a needed location within a body cavity, after which, in step 910, the endoscope handle manipulator can cause execution of the specified type of endoscopic procedure selected above. In step 912, either the surgeon or the autonomous system can evaluate the completed step and determine if further work needs to be done or if additional work still needs to be completed, and can then direct the controller to execute a new procedure.

In a further embodiment, the method for performing an endoscopic procedure may include the additional steps of using sensors to obtain location and position data to navigate the endoscope into a needed position. In a still further step, imaging data from the sensors may also be used to analyze potential pathologies and determine if additional diagnostic procedures or treatment procedures should be performed. The endoscope may then be automatically reconfigured with the appropriate tools to perform the needed diagnostic or treatment procedures.

These methods described above may be incorporated into methods for automated screening, diagnostic and treatment of a multitude of pathologies, including colon or pancreatic cancer.

### Computer-enabled Embodiment

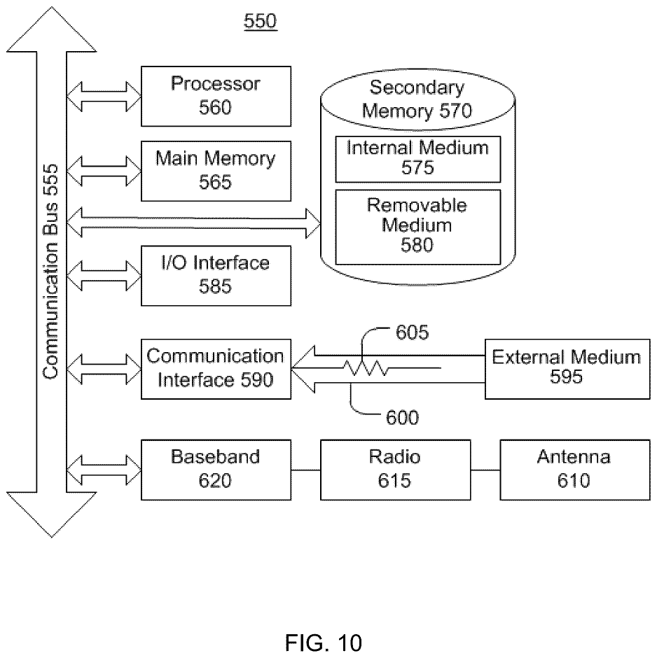

FIG. 10 is a block diagram illustrating an example wired or wireless system 550 that may be used in connection with various embodiments described herein. For example, the system 550 may be used as or in conjunction with the device and methods as previously described with respect to FIGS. 1-9. The system 550 can be a conventional personal computer, computer server, personal digital assistant, smart phone, tablet computer, or any other processor enabled device that is capable of wired or wireless data communication. Other computer systems and/or architectures may be also used, as will be clear to those skilled in the art.

The system 550 preferably includes one or more processors, such as processor 560. Additional processors may be provided, such as an auxiliary processor to manage input/output, an auxiliary processor to perform floating point mathematical operations, a special-purpose microprocessor having an architecture suitable for fast execution of signal processing algorithms (e.g., digital signal processor), a slave processor subordinate to the main processing system (e.g., back-end processor), an additional microprocessor or controller for dual or multiple processor systems, or a coprocessor. Such auxiliary processors may be discrete processors or may be integrated with the processor 560.

The processor 560 is preferably connected to a communication bus 555. The communication bus 555 may include a data channel for facilitating information transfer between storage and other peripheral components of the system 550. The communication bus 555 further may provide a set of signals used for communication with the processor 560, including a data bus, address bus, and control bus (not shown). The communication bus 555 may comprise any standard or non-standard bus architecture such as, for example, bus architectures compliant with industry standard architecture (“ISA”), extended industry standard architecture (“EISA”), Micro Channel Architecture (“MCA”), peripheral component interconnect (“PCI”) local bus, or standards promulgated by the Institute of Electrical and Electronics Engineers (“IEEE”) including IEEE 488 general-purpose interface bus (“GPIB”), IEEE 696/S-100, and the like.

System 550 preferably includes a main memory 565 and may also include a secondary memory 570. The main memory 565 provides storage of instructions and data for programs executing on the processor 560. The main memory 565 is typically semiconductor-based memory such as dynamic random access memory (“DRAM”) and/or static random access memory (“SRAM”). Other semiconductor-based memory types include, for example, synchronous dynamic random access memory (“SDRAM”), Rambus dynamic random access memory (“RDRAM”), ferroelectric random access memory (“FRAM”), and the like, including read only memory (“ROM”).

The secondary memory 570 may optionally include a internal memory 575 and/or a removable medium 580, for example a floppy disk drive, a magnetic tape drive, a compact disc (“CD”) drive, a digital versatile disc (“DVD”) drive, etc. The removable medium 580 is read from and/or written to in a well-known manner. Removable storage medium 580 may be, for example, a floppy disk, magnetic tape, CD, DVD, SD card, etc.

The removable storage medium 580 is a non-transitory computer readable medium having stored thereon computer executable code (i.e., software) and/or data. The computer software or data stored on the removable storage medium 580 is read into the system 550 for execution by the processor 560.

In alternative embodiments, secondary memory 570 may include other similar means for allowing computer programs or other data or instructions to be loaded into the system 550. Such means may include, for example, an external storage medium 595 and an interface 570. Examples of external storage medium 595 may include an external hard disk drive or an external optical drive, or and external magneto-optical drive.

Other examples of secondary memory 570 may include semiconductor-based memory such as programmable read-only memory (“PROM”), erasable programmable read-only memory (“EPROM”), electrically erasable read-only memory (“EEPROM”), or flash memory (block oriented memory similar to EEPROM). Also included are any other removable storage media 580 and communication interface 590, which allow software and data to be transferred from an external medium 595 to the system 550.

System 550 may also include an input/output (“I/O”) interface 585. The I/O interface 585 facilitates input from and output to external devices. For example the I/O interface 585 may receive input from a keyboard or mouse and may provide output to a display. The I/O interface 585 is capable of facilitating input from and output to various alternative types of human interface and machine interface devices alike.

System 550 may also include a communication interface 590. The communication interface 590 allows software and data to be transferred between system 550 and external devices (e.g. printers), networks, or information sources. For example, computer software or executable code may be transferred to system 550 from a network server via communication interface 590. Examples of communication interface 590 include a modem, a network interface card (“NIC”), a wireless data card, a communications port, a PCMCIA slot and card, an infrared interface, and an IEEE 1394 fire-wire, just to name a few.

Communication interface 590 preferably implements industry promulgated protocol standards, such as Ethernet IEEE 802 standards, Fiber Channel, digital subscriber line (“DSL”), asynchronous digital subscriber line (“ADSL”), frame relay, asynchronous transfer mode (“ATM”), integrated digital services network (“ISDN”), personal communications services (“PCS”), transmission control protocol/Internet protocol (“TCP/IP”), serial line Internet protocol/point to point protocol (“SLIP/PPP”), and so on, but may also implement customized or non-standard interface protocols as well.

Software and data transferred via communication interface 590 are generally in the form of electrical communication signals 605. These signals 605 are preferably provided to communication interface 590 via a communication channel 600. In one embodiment, the communication channel 600 may be a wired or wireless network, or any variety of other communication links. Communication channel 600 carries signals 605 and can be implemented using a variety of wired or wireless communication means including wire or cable, fiber optics, conventional phone line, cellular phone link, wireless data communication link, radio frequency (“RF”) link, or infrared link, just to name a few.

Computer executable code (i.e., computer programs or software) is stored in the main memory 565 and/or the secondary memory 570. Computer programs can also be received via communication interface 590 and stored in the main memory 565 and/or the secondary memory 570. Such computer programs, when executed, enable the system 550 to perform the various functions of the present invention as previously described.

In this description, the term “computer readable medium” is used to refer to any non-transitory computer readable storage media used to provide computer executable code (e.g., software and computer programs) to the system 550. Examples of these media include main memory 565, secondary memory 570 (including internal memory 575, removable medium 580, and external storage medium 595), and any peripheral device communicatively coupled with communication interface 590 (including a network information server or other network device). These non-transitory computer readable mediums are means for providing executable code, programming instructions, and software to the system 550.

In an embodiment that is implemented using software, the software may be stored on a computer readable medium and loaded into the system 550 by way of removable medium 580, I/O interface 585, or communication interface 590. In such an embodiment, the software is loaded into the system 550 in the form of electrical communication signals 605. The software, when executed by the processor 560, preferably causes the processor 560 to perform the inventive features and functions previously described herein.

The system 550 also includes optional wireless communication components that facilitate wireless communication over a voice and over a data network. The wireless communication components comprise an antenna system 610, a radio system 615 and a baseband system 620. In the system 550, radio frequency (“RF”) signals are transmitted and received over the air by the antenna system 610 under the management of the radio system 615.

In one embodiment, the antenna system 610 may comprise one or more antennae and one or more multiplexors (not shown) that perform a switching function to provide the antenna system 610 with transmit and receive signal paths. In the receive path, received RF signals can be coupled from a multiplexor to a low noise amplifier (not shown) that amplifies the received RF signal and sends the amplified signal to the radio system 615.

In alternative embodiments, the radio system 615 may comprise one or more radios that are configured to communicate over various frequencies. In one embodiment, the radio system 615 may combine a demodulator (not shown) and modulator (not shown) in one integrated circuit (“IC”). The demodulator and modulator can also be separate components. In the incoming path, the demodulator strips away the RF carrier signal leaving a baseband receive audio signal, which is sent from the radio system 615 to the baseband system 620.

If the received signal contains audio information, then baseband system 620 decodes the signal and converts it to an analog signal. Then the signal is amplified and sent to a speaker. The baseband system 620 also receives analog audio signals from a microphone. These analog audio signals are converted to digital signals and encoded by the baseband system 620. The baseband system 620 also codes the digital signals for transmission and generates a baseband transmit audio signal that is routed to the modulator portion of the radio system 615. The modulator mixes the baseband transmit audio signal with an RF carrier signal generating an RF transmit signal that is routed to the antenna system and may pass through a power amplifier (not shown). The power amplifier amplifies the RF transmit signal and routes it to the antenna system 610 where the signal is switched to the antenna port for transmission.

The baseband system 620 is also communicatively coupled with the processor 560. The central processing unit 560 has access to data storage areas 565 and 570. The central processing unit 560 is preferably configured to execute instructions (i.e., computer programs or software) that can be stored in the memory 565 or the secondary memory 570. Computer programs can also be received from the baseband processor 610 and stored in the data storage area 565 or in secondary memory 570, or executed upon receipt. Such computer programs, when executed, enable the system 550 to perform the various functions of the present invention as previously described. For example, data storage areas 565 may include various software modules (not shown) that are executable by processor 560.

Various embodiments may also be implemented primarily in hardware using, for example, components such as application specific integrated circuits (“ASICs”), or field programmable gate arrays (“FPGAs”). Implementation of a hardware state machine capable of performing the functions described herein will also be apparent to those skilled in the relevant art. Various embodiments may also be implemented using a combination of both hardware and software.

Furthermore, those of skill in the art will appreciate that the various illustrative logical blocks, modules, circuits, and method steps described in connection with the above described figures and the embodiments disclosed herein can often be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a module, block, circuit or step is for ease of description. Specific functions or steps can be moved from one module, block or circuit to another without departing from the invention.

Moreover, the various illustrative logical blocks, modules, and methods described in connection with the embodiments disclosed herein can be implemented or performed with a general purpose processor, a digital signal processor (“DSP”), an ASIC, FPGA or other programmable logic device, discrete gate or transistor logic, discrete hardware components, or any combination thereof designed to perform the functions described herein. A general-purpose processor can be a microprocessor, but in the alternative, the processor can be any processor, controller, microcontroller, or state machine. A processor can also be implemented as a combination of computing devices, for example, a combination of a DSP and a microprocessor, a plurality of microprocessors, one or more microprocessors in conjunction with a DSP core, or any other such configuration.

Additionally, the steps of a method or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of storage medium including a network storage medium. An exemplary storage medium can be coupled to the processor such the processor can read information from, and write information to, the storage medium. In the alternative, the storage medium can be integral to the processor. The processor and the storage medium can also reside in an ASIC.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

What is claimed is:

1. A robotically-controlled dual-arm flexible endoscope, comprising:
a flexible endoscope with a shaft, a proximal handle and a distal tip;
a first robotic arm secured with the proximal handle and configured to move the proximal handle of the flexible endoscope;
an endoscope handle manipulator connected with the first robotic arm and proximal handle, and configured to control the flexible endoscope;
a second robotic arm with an endoscope actuator configured to secure the distal tip and advance or withdrawal the flexible endoscope into or from a body cavity, respectively; and
a controller in communication with the first robotic arm and second robotic arm to control the movement and position of each robotic arm, the handle manipulator and the endoscope actuator.

2. The endoscope of claim 1, wherein the first robotic arm controls a location, movement and torque of the endoscope handle manipulator at a proximal end of the flexible endoscope.

3. The endoscope of claim 2, wherein the second robotic arm controls a location, movement and torque of the endoscope actuator to advance or withdrawal the flexible endoscope into or from the body cavity.

4. The endoscope of claim 3, wherein the first robotic arm and second robotic arm provide six degrees of freedom for the flexible endoscope.

5. The endoscope of claim 1, wherein the controller is autonomously controlled by a computing device to control the movement and position of each robotic arm, the handle manipulator and the endoscope actuator.

6. The endoscope of claim 5, wherein computing device utilizes computer vision and machine learning to control the movement and position of each robotic arm, the handle manipulator and the endoscope actuator.

7. The endoscope of claim 1, wherein flexible endoscope further comprises a scope shaft with at least one sensor embedded therein.

8. The endoscope of claim 7, wherein the scope shaft includes a plurality of sensors disposed along the length of the scope shaft.

9. The endoscope of claim 8, wherein the plurality of sensors determine at least one of a position, location and movement of the scope shaft within a body cavity.

10. The endoscope of claim 1, further comprising a rail disposed along an inner circumference of the scope shaft configured to deliver at least one endoscopic tool to a distal end of the endoscope.

11. A method of performing an endoscopic procedure with a robotically-controlled flexible endoscope, the method comprising:
controlling, via a controller, the movement and function of an endoscope handle manipulator connected with a proximal handle of a flexible endoscope via a first robotic arm;
controlling, via a controller, the movement and function of an endoscope actuator secured with a distal tip of the flexible endoscope via a second robotic arm; and
performing the endoscopic procedure by via movement and control of the flexible endoscope by the controller.

12. The method of claim 11, further comprising the controller controlling a location, movement and torque of the endoscope handle manipulator at a proximal end of the flexible endoscope using the first robotic arm.

13. The method of claim 12, further comprising the controller controlling a location, movement and torque of the endoscope actuator to advance or withdrawal the flexible endoscope into or from the body cavity using the second robotic arm.

14. The method of claim 13, further comprising providing six degrees of freedom of movement of the flexible endoscope utilizing the first robotic arm and second robotic arm.

15. The method of claim 11, further comprising autonomously controlling the controller using a computing device.

16. The method of claim 15, further comprising utilizing computer vision and machine learning to control the movement and position of each robotic arm, the handle manipulator and the endoscope actuator.

17. The method of claim 11, further comprising determining at least one of a position, location and movement of a scope shaft within a body cavity using a plurality of sensors disposed along a length of the scope shaft.

18. The method of claim 11, further comprising delivering at least one endoscopic tool to a distal end of the endoscope using a rail disposed along an inner circumference of the scope shaft.

19. A method of screening for a disease using an autonomous flexible endoscope, the method comprising:
inserting a flexible endoscope into a body cavity using a first robotic arm and second robotic arm controlled by a controller to adjust a movement and function of the flexible endoscope;
utilizing a plurality of sensors disposed along the endoscope and at a distal end to identify pathologies which indicate a presence of disease;
conducting at least one diagnostic test using the plurality of sensors and at least one tool disposed at the distal end of the endoscope to determine the presence of the disease; and
performing at least one endoscopic surgical procedure to treat the identified disease.

20. The method of claim 19, wherein the method of screening for the disease is one of colon cancer or pancreatic cancer.
